(19) United States
(12) Patent Application Publication
(10) Pub. No.: US 2014/0116647 A1
(43) Pub. Date: May 1, 2014

(54) SCOPE CRADLE

(71) Applicants: Bruce Kannry, Naperville, IL (US); Christopher Olson, Naperville, IL (US)

(72) Inventors: Bruce Kannry, Naperville, IL (US); Christopher Olson, Naperville, IL (US)

(21) Appl. No.: 14/068,898

(22) Filed: Oct. 31, 2013

Related U.S. Application Data
(60) Provisional application No. 61/720,869, filed on Oct. 31, 2012.

(51) Int. Cl. F28F 9/00; A61B 19/02

(52) U.S. Cl. CPC F28F 9/005; A61B 19/0256
USPC 165/80.5

(57) ABSTRACT

A device holds at least one medical instrument in a fluid bath intended for maintaining the temperature of the instrument. The device includes a first portion disposed at one end of the bath, the first portion including at least one well adapted to hold the instrument relative to the bath. The device also includes a second portion connected to the first portion at the one end of the bath, whereby the first portion is maintained in a position to hold the instrument securely in the bath.

### BACKGROUND

The present invention relates generally to a device for holding medical instruments, including, for example a cradle that safely stores laparoscopes in a fluid bath during a surgical procedure.

Laparoscopy is performed to examine the abdominal and pelvic organs to diagnose certain conditions and—depending on the condition—can be used to perform surgery. The use of laparoscopes in abdominal and pelvic surgery is popular because laparoscopes allow a surgeon to perform complex, minimally invasive surgical techniques while significantly reducing a patient's pain, discomfort, and recovery time resulting from large incisions made during typical open surgery.

The fogging of laparoscopic lenses during surgery can lead to costly delays. Fluid bath systems to warm scopes and prevent lens fogging are known. A bath capable of maintaining hot water at a stable temperature (such as 120° F.) is an effective technique for maintaining warm laparoscope lenses and preventing lens fogging during laparoscopy. In the past, laparoscopes placed in fluid baths often rested on the upper edges of the baths and were prone to tipping over or falling out of the bath, as the bulk of the weight of the laparoscope is disposed in its head portion, while the arm of the scope carries comparatively much less weight. Such incidents often lead to costly damage of the laparoscopes, which are expensive precision instruments.

### SUMMARY

In one embodiment, the invention provides a cradle that securely cradles medical instruments, such as robotic scopes, placed in a fluid warming system, thereby protecting the scopes from accidental tipping and falling. The cradle ensures that expensive robotic equipment remains safe and secure during surgery or other medical procedures.

Other aspects of the invention will become apparent by consideration of the detailed description and accompanying drawings.

### BRIEF DESCRIPTION OF THE DRAWINGS

FIG. 1a is a front elevational view of a scope cradle according to an exemplary embodiment.

FIG. 1b is a side elevational view of the cradle of FIG. 1a.

FIG. 1c is a top view of the cradle of FIG. 1a.

FIG. 1d is a bottom view of the cradle of FIG. 1a.

FIG. 2 is a perspective view of a cradle secured to a fluid bath.

FIG. 3 is a perspective view of laparoscopes resting in a cradle.

FIG. 4 is an alternative embodiment of the cradle.

FIG. 5 is another alternative embodiment of the cradle.

### DETAILED DESCRIPTION

It is to be understood that the invention is not limited in its application to the details of construction and the arrangement of components set forth in the following description or illustrated in the following drawings. The invention is capable of other embodiments and of being practiced or of being carried out in various ways.

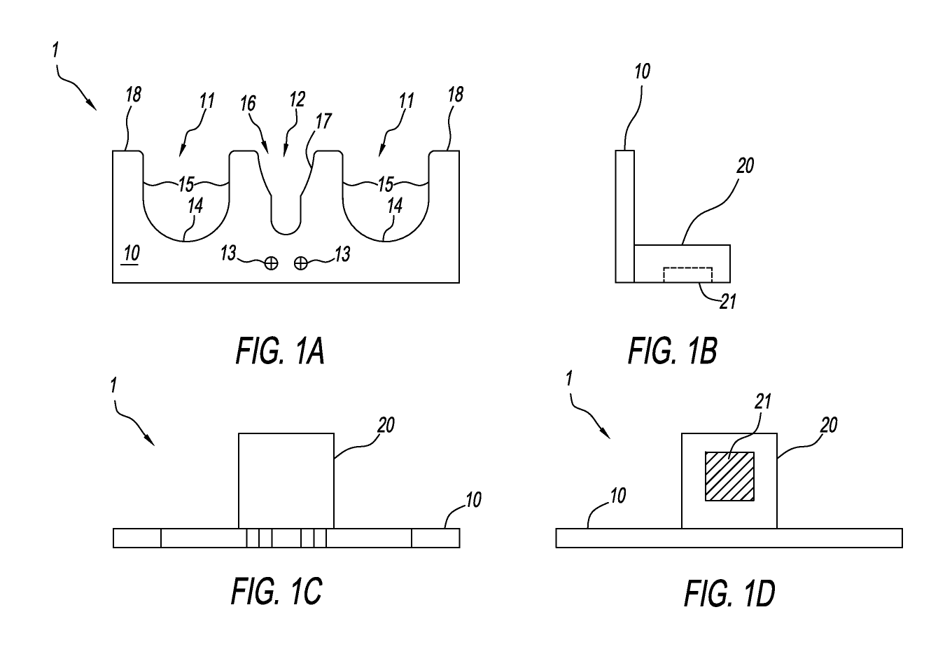

A device comprising a cradle 1 (hereinafter “scope cradle” or “cradle”) for holding a laparoscope 40 is shown in FIG. 1. Although the term laparoscope is used, it will be understood that the cradle of the present invention can be used to hold other types of medical instruments, such as endoscopes and forceps. The illustrated scope cradle 1 includes a first or upright portion 10, which is adjacent an end of a fluid bath 30, and a second portion or block 20 disposed adjacent the upright portion 10 to support the upright portion. The upright portion 10 includes two larger, outer cradle portions or “wells” 11 and a smaller, central well 12. The three wells 11, 12 provide the cradle with the ability to store and warm multiple scopes 40 in a single fluid bath.

As shown in FIG. 1, the upright portion 10 is connected to the block 20, with the resulting connection leaving the upright portion 10 and block 20 generally perpendicular to each other. The upright portion 10 may be connected to the block 20 through a number of ways. One form of connection is a mechanical connection using bolts or screws. Two bolt holes 13 in the upright portion 10 are shown in FIG. 1a. As shown in FIGS. 1b and 1d, a magnet 21 may be embedded within a lower surface of the block 20 using an adhesive or a snap-fit connection. In an exemplary embodiment, an adhesive known in the art for use on steel, aluminum, or thermoplastics such as Loctite® is used. Alternatively, the upright portion 10 and the block 20 may be formed integral with each other as a single piece.

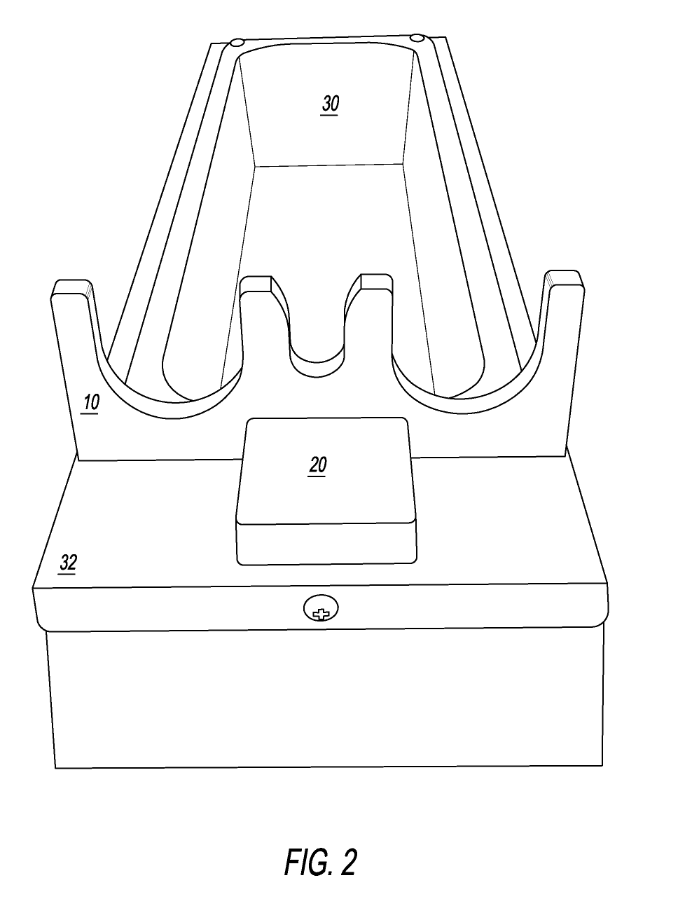

Referring to FIG. 2, the magnet 21 allows the block 20 (and the overall scope cradle 1), which may be formed of a non-magnetic material (such as aluminum), to be rigidly secured to the outer casing of the fluid bath 30, which is typically formed of a magnetic material (such as sheet metal). In an exemplary embodiment, the magnet is made of neodymium, and may also include iron and boron. Such magnets have demonstrated superior performance at high temperatures.

The upright portion 10 and the block 20 of the scope cradle 1 are generally formed of a metal, such as aluminum or stainless steel. However, the upright portion 10 and block 20 may be formed from other materials, including plastics, polycarbonates, or LEXAN®. The inner sides of the wells 11, 12 may optionally include a material lining so as to soften the surface on which the wells receive the scopes. The lining may be formed of cloth, a soft plastic, or a sponge-like material.

Referring to FIG. 1 a, the two wells 11 have a concave shape and provide a platform 14 on which a head portion 41 of the scope 40 may rest while the body 42 of the scope 40 is disposed in the fluid bath 30. In FIG. 1a, the wells 11 generally have a “U” shape. However, other shapes are possible. The size and shape of the concavity of the wells 11 is variable, depending on the type of scope to be held by the scope cradle 1. In an exemplary embodiment, the wells 11 are sized to hold 0° and 30° robotic laparoscopes 40, while the well 12 is sized to hold a traditional 5 mm scope 40.

The upright walls 15 of the wells 11 form side guards that prevent scopes 40 from rolling laterally or tipping. As shown in FIG. 1a, the opening 16 of the well 12 may include a portion 17 that flares outward near the top surface 18 of the upright portion 10 to allow scopes 40 to be more easily placed within the well 12. Providing a wider opening at the top of the well 12 also permits the lower part of the well to be narrower so as to better stabilize the scope 40 within the well 12. The openings of the wells 11 may also include flared portions.

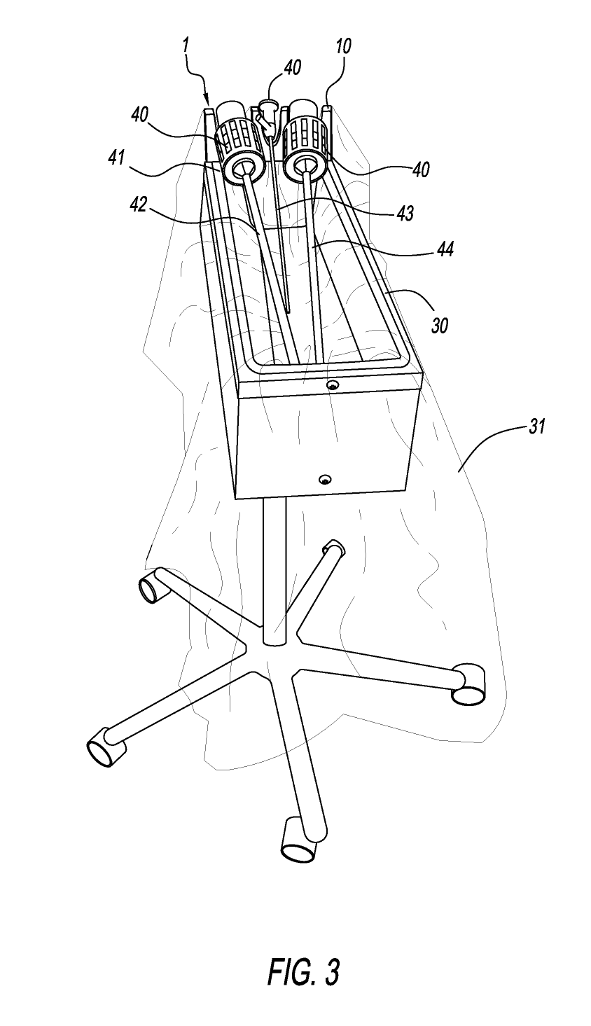

A scope cradle 1 is shown in combination with a fluid bath 30 in FIG. 2. The fluid bath 30 may be formed of a metal such as stainless steel, as is known in the art. The scope cradle 1 may be secured to the fluid bath 30 mechanically, magnetically, or may be formed integral with the fluid bath 30. In the embodiment of FIGS. 1-3, the scope cradle 1 is secured magnetically to the frame or case 32 of the fluid bath 30 via the magnet 21 embedded in the block 20. As shown in FIG. 3, the scope cradle 1 is positioned adjacent to one end of the fluid bath one so that the bodies 42, 43, 44 of the scopes can easily rest within the bath 30.

As shown in FIG. 3, the fluid bath 30 may optionally include a sterile plastic sheet 31, as is generally known in the art. The sheet 31 may be overlaid onto the fluid bath 30 and the wells 11, 12, with the scopes 40 being placed on top of the plastic sheet 31 so as to not directly contact the fluid in the fluid bath 30.

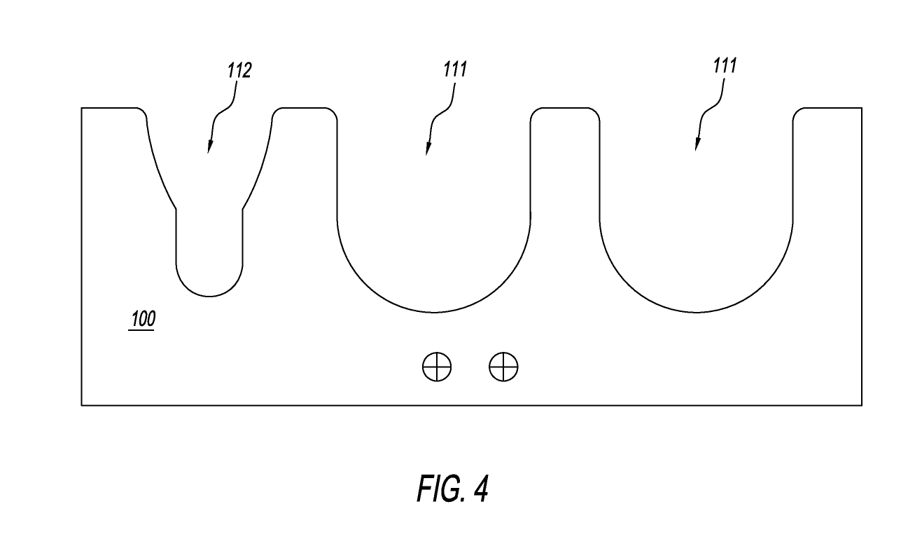

An alternative embodiment of the scope cradle 100, illustrated in FIG. 4, includes a smaller well 112 located adjacent one lateral end of the cradle 100 and two larger wells 111 located in the middle and adjacent the other lateral end, respectively.

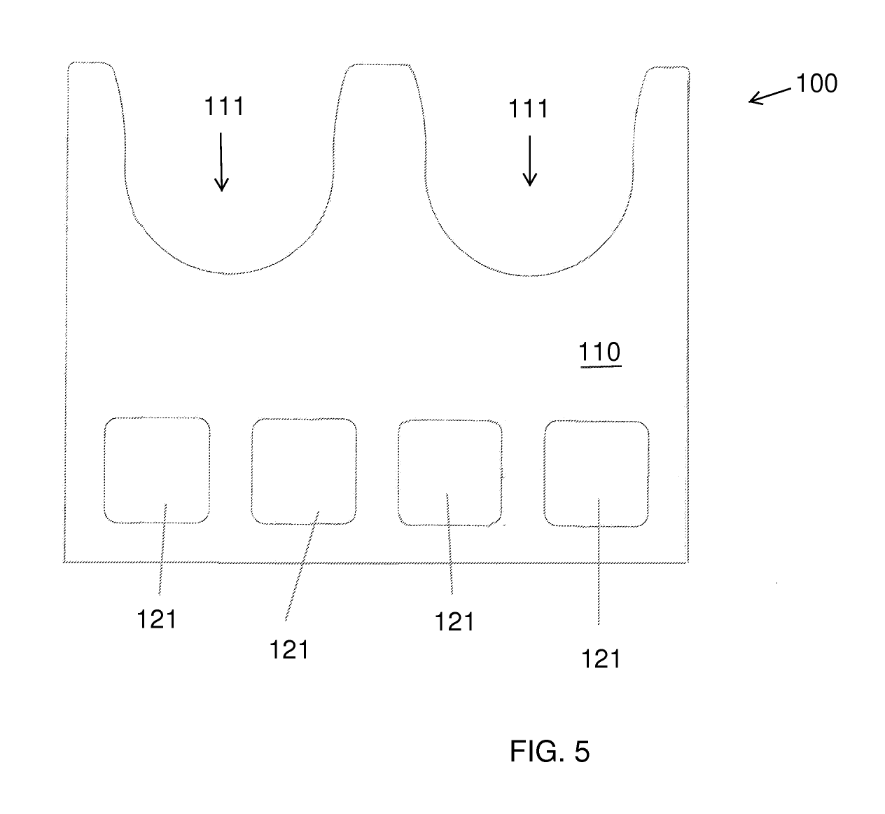

While FIG. 2 shows a single scope cradle 1 at one longitudinal end of the bath 30, a second scope cradle 100 as shown in FIG. 5 may be disposed at the opposite longitudinal end, thereby increasing the number of scopes 40 to be heated in the bath 30 at a given time. The cradle 100 is similar in structure to the cradle 1 and includes a first or upright portion 110 and wells 111. The cradle 100 further includes magnets 121 disposed beneath the wells 111 that may be used, for example, to attach the cradle 100 to a side wall of the bath 30. In such a configuration, the upright portion 110 of the cradle 100 would face the upright portion 10 of the cradle 1. Alternatively, the cradle 100 may be the only cradle positioned on the bath 30.

1. A device for holding at least one medical instrument in a fluid bath intended for maintaining the temperature of the instrument, the device comprising:
a first portion disposed at one end of the bath, the first portion including at least one well adapted to hold the instrument relative to the bath, and a second portion connected to the first portion at the one end of the bath, whereby the first portion is maintained in a position to hold the instrument securely in the bath.

2. The device of claim 1, wherein the first portion includes a plurality of wells for holding a plurality of medical instruments.

3. The device of claim 2, wherein the plurality of wells includes a pair of outer wells and at least one inner well disposed between the outer wells.

4. The device of claim 3, wherein at least one of the outer wells is larger than the inner well.

5. The device of claim 2, wherein at least two of the wells have different sizes from each other.

6. The device of claim 2, wherein the wells are parallel with each other along a length of the first portion.

7. The device of claim 2, wherein at least one of the wells extends through an entire thickness of the first portion.

8. The device of claim 2, wherein the wells are generally U-shaped.

9. The device of claim 7, wherein ends of the U-shaped wells are flared such that an upper portion of the well is wider than a lower portion of the well.

10. The device of claim 1, wherein at least one of the first and second portions is formed of metal.

11. The device of claim 1, wherein at least one of the first and second portions is formed of plastic.

12. The device of claim 1, wherein the second portion includes a magnet for attaching the device to the fluid bath.

13. The device of claim 1, wherein inner sides of at least one of the wells includes a material lining to soften a surface on which the instrument is to be received.

14. The device of claim 11, wherein the magnet includes neodymium, iron, or boron.

15. The device of claim 1, wherein the medical instrument is a robotic laparoscope.

16. The device of claim 1, wherein at least one of the wells is sized to hold a 0° or 30° robotic laparoscope therein.

17. The device of claim 1, wherein the first and second portions are integral with each other.

18. The device of claim 1, wherein the first and second portions are separate pieces, which are attached by screws, bolts, or adhesive.

19. The device of claim 1, wherein the first and second portions are oriented generally perpendicular to each other.

20. The device of claim 1, wherein the well extends generally perpendicular to a length of the bath.

21. A fluid bath for holding at least one medical instrument to maintain the temperature of the instrument, the bath comprising:
a first device including a first portion disposed at one end of the bath, the first portion including at least one well adapted to hold the instrument relative to the bath, and a second portion connected to the first portion at the one end of the bath, whereby the first portion is maintained in a position to hold the instrument securely in the bath; and
a second device including a third portion disposed opposite the one end of the bath, the third portion including at least one well adapted to hold the instrument relative to the bath, whereby the third portion is maintained in a position to hold the instrument securely in the bath.
